(19) United States
(12) Patent Application Publication
(10) Pub. No.: US 2004/0219537 A1
(43) Pub. Date: Nov. 4, 2004

(54) Epidermal collection method, kit, and device

(76) Inventors: Richard K. Fenrich, Blacksburg, VA (US); Matthew Rarick, Blacksburg, VA (US); Susannah Wurgler-Murphy, Blacksburg, VA (US); David R. Comeau, Sterling, MA (US)

Correspondence Address: Anthony P. Venturino, Suite 850, 1615 L Street N.W., Washington, DC, 20036, US

(21) Appl. No.: 10/427,985

(22) Filed: May 2, 2003

(51) Int. Cl. C12Q 1/68

(52) U.S. Cl. 435/6; 435/40.5

(57) ABSTRACT

The present invention discloses a method, device, and kit to non-invasively sample epidermal cells of a test subject while ensuring the collection of a large enough quantity of cells for subsequent biological testing. In particular, the method obtains human, or other living creature's, nucleic acid or other biological component for cytological or diagnostic testing and/or genetic analysis. The method takes the sample of the epidermis by means of an abrading surface which scrapes off and retains cell samples for subsequent characterization. The abrading surface could be single use, in a sealed container, where the sample can be returned without contamination. These nucleic acids can be either eluted or resolubilized off, or otherwise released after collection or storage, in a manner that enables them to be amplified by conventional amplification methods such as polymerase chain reaction. Also provided are devices for storing DNA and a kit for easily obtaining and stably storing a sample of epidermal cells and/or other DNA bearing material.

### FIELD OF THE INVENTION

The present invention discloses a method, device, and kit to non-invasively sample epidermal cells, or other DNA bearing material taken from the surface of the epidermis, for example oils or sweat, of a test subject while ensuring the collection of a large enough quantity of cells or other material for subsequent biological analyses. In particular, the method obtains cells by taking a sample of the epidermis by means of an abrading and/or cutting surface that scrapes off and retains cell samples for subsequent genetic characterization. The abrading and/or cutting surface would be single use, in a sealed container, where the sample can be returned without contamination. Cellular material, such as DNA (deoxyribonucleic acid), can be released from the collected cells in a manner that enables the cellular material to be analyzed by biological methods. For example, DNA could be amplified by a method such as the polymerase chain reaction. Also provided are devices for taking and storing the epidermal sample and a kit for taking and storing the epidermis. The present invention allows an epidermal sample to be easily obtained and stably stored for a long period of time. In the present specification the term “cells” encompasses free cells and/or tissue that are collections of cells with or without intercellular bonds.

### BACKGROUND OF THE INVENTION

Genetic identification, including DNA profiling methods, can discern one individual from another. By virtue of its accuracy, genetic analysis is regarded as the best tool for identification in fields such as forensics, medical diagnostics and genealogy (paternity). In practice, in the U.K. and the U.S., DNA profile databases for prisoners have been constructed. These databases contain DNA profiles of criminals and evidence obtained at crime scenes. Using a computer, the known profiles in these databases are compared against unknown profiles from crime scene evidence.

Advancement of DNA analysis techniques will lead to a large variety of tests for genetic diseases and predispositions in the near future. One of the limitations of almost all existing tests is that an invasive or offensive procedure must be employed to obtain a sample for subsequent analyses. Some examples of current sampling methods include blood withdrawal, hair pulling, or buccal swabbing. Furthermore, many of these methods can expose medical personnel to infectious agents. Though these techniques are effective, test subjects would prefer a completely non-invasive, painless, and non-offensive test.

Many times a long-term storage solution is needed to maintain the integrity of the sample once the cells have been collected. Whatman and Schleicher & Schuell have both been granted patents regarding long term storage of DNA bearing material. Whatman uses chemically treated paper to deactivate and destroy microorganisms while attaching the DNA permanently to the paper. See U.S. Pat. Nos. 6,322,983, 6,294,203, 5,985,327, 5,976,572, 5,972,386, 5,756,126, all of which are incorporated herein by reference, as well as www.whatman.com. Schleicher & Schuell, also chemically treats paper to collect, store, and purify the DNA sample. See U.S. Pat. Nos. 6,168,922 and 5,939,259, both of which are incorporated herein by reference, as well as the homepage www.s-and-s.com. Both methods described above require that the sample be applied to the paper in a liquid form.

WO 97/18009, incorporated herein by reference, discloses a system and a kit for non-invasive collection of DNA-bearing material from the inner cheeks of a living person or corpse. This includes swabs of high modulus fibers that scrape DNA material from the inner cheeks to obtain sufficient material for future identification. It specifically teaches away from sampling cells from skin. It also mentions a kit that includes the swabs and a means for obtaining and recording ink fingerprints.

U.S. Pat. No. 6,355,439 Chung, et al., incorporated herein by reference, discloses a method for obtaining human DNA for genetic analysis, by taking a sample of the epidermis of test subject by means of an adhesive sheet, and by extracting DNA from the epidermis stuck on the adhesive sheet. It also discloses combined sheets for storing DNA and a kit for taking the epidermis. In addition, a figured epidermal print for fingerprint identification and the epidermal scraps for subsequent DNA analysis of a test subject can be taken at the same time.

U.S. Pat. No. 5,879,326 to Godshall et al., incorporated herein in its entirety by reference, discloses a method and apparatus for mechanically disrupting a layer of skin having a known thickness without substantially disrupting underlying dermis layers below the skin to facilitate the delivery of compounds across the disrupted layer.

U.S. Pat. No. 5,732,719 to Godbout, incorporated herein by reference, discloses a filing implement comprising a strip of fiberglass with embedded diamond particles having a grit ranging from 50 to 500 mesh.

U.S. Pat. No. 6,168,922 to Harvey, et al., incorporated herein by reference, discloses devices and methods for the collection, storage, and purification of nucleic acids, such as DNA or RNA, from fluid samples for subsequent genetic characterization, primarily by conventional amplification methods. It can be used to collect, store, or purify nucleic acids either from a biological source other than untreated whole blood, the biological source having naturally occurring nucleic acid amplification inhibitors present, (including either a buccal swab, cerebrospinal fluid, feces, lymphatic fluid, a plasma sample, a saliva sample, a serum sample, urine, or a suspension of cells or viruses), or from a treated whole blood source that has naturally occurring nucleic acid amplification inhibitors present, as well as added blood stabilization components that also inhibit nucleic acid amplification. In particular, an absorbent material that does not bind nucleic acids irreversibly is impregnated with a chaotropic salt. A biological source sample is contacted with the impregnated absorbent material. Any nucleic acids present in the biological source can be either eluted or resolubilized off the absorbent material.

U.S. Pat. No. 2,835,246 to Boettger, incorporated herein by reference discloses a medical specimen handling device which is shaped as a spoon or has a head which is grooved for the direct taking of swabs. In this device, the sample collection segment folds back or detaches to be stored in the container which also acts as the handle for the sample collection segment. This device is designed for collection and secure handling of liquid medical specimens and is not suitable for scraping and retention of epidermal samples.

U.S. Pat. No. 4,981,143 to Sakita, et al., incorporated herein by reference, discloses a cell sampler designed to obtain cells, particularly cells from the cervical canal of the uterus, for cytology, by scraping cells from a soft tissue area. The cell sampler has a stick shaped abrading segment having a number of edges adapted to scrape off cells from the cervical canal. There is a conical/pyramidal stick shaped abrading segment and a spatula shaped abrading segment. The abrading segment tapers to a reduced width to aid in insertion into the body cavity to be sampled. The thin handles and tapered design of these devices make them unsuitable for collection of skin scrapings which require the application of significant pressure. These devices do not provide for secure storage of the collected sample.

U.S. Pat. No. 5,137,030 issued to Darougar, incorporated herein by reference, discloses a slotted probe for collecting mucous tissue scrapings into the slots. This device is designed for sampling soft tissue and does not provide for secure storage of the collected sample.

There is a need for sampling devices and methods to painlessly sample skin cells in sufficient quantity for nucleic acid, for example DNA or RNA analysis, or other biological analyses, for example, cytological or diagnostic analysis. There is also a need for improved devices and methods for long-term storage of DNA bearing samples.

### SUMMARY OF THE INVENTION

The present invention provides a method, apparatus, and kit to painlessly sample skin cells in sufficient quantity for DNA analysis or for analyses other than DNA analysis, for example, cytological or other scientific analyses, from humans or animals or other life forms.

The present invention permits sampling epidermal cells from animals, including endangered species without killing or permanently harming them. For example, cells could be sampled from the footpads of such animals.

This invention provides an apparatus that loosens, scrapes, and retains epidermal cell samples from the epidermis of a test subject. Typical apparati include bladed devices wherein the blade top wall optionally has additional abrasive and/or adhesive.

The “epidermis” (used herein interchangeably with the term “epidermal skin”) is defined as external (surface) skin not including interior body or body cavity tissues. The present sampling method collects epidermal cells which are relatively dry upon collection (of course the water naturally in the cells and incidental moisture which may be present on the skin at the time of collection are permitted). This contrasts with collection of blood or buccal cells which, by nature, are suspended in liquid. The lack of suspending water addresses the concern of DNA degradation by microorganisms. Reduced water content inhibits the growth of bacteria, yeasts and molds. To further ensure the integrity of the sample, the sampling device can either be covered with a cap with or without a desiccant or placed inside a vapor barrier bag, with or without desiccant. The bag can then be sealed.

The present invention also provides improved devices and methods for long-term storage of DNA bearing samples.

The device is envisioned as single use but it could also be designed for multiple uses. When a cell sample has been taken, the portion of the device containing the sampled tissue and cells is sealed and stored for later use as appropriate. The sample is covered with a tape or other cover to prevent contamination. For longer term storage, a desiccant can be used to maintain a controlled low humidity environment. The desiccant, which can be placed in the cover in some instances, or inside a vapor barrier bag in other instances, will reduce moisture required for viability of microorganisms. In the latter case, the entire sampling device would be placed in a vapor barrier bag containing a desiccant and the bag would be heat or otherwise sealed to maintain the integrity of the sample over a longer time period. Maintaining the sample in a controlled humidity environment provides for long-term storage of the sample.

In the present specification the term “loosen” means mechanical, chemical, or other means of breaking bonds between cells so that a cell or group of cells is freed from the original structure to which the cell or group of cells was bonded. Examples of loosening means include an abrading device having an abrading surface, enzymes such as, but not restricted to, papain to break bonds between cells, or devices which generate sonic energy to break cell bonds. As used in the present specification an abrading surface (also termed in the present specification as an “abrading or cutting” surface) to abrade the surface of the skin encompasses an abrasive or cutting surface that causes mechanical disruption of the epidermis. Typical abrading surfaces include a knife-like or blade-like instrument, sandpaper, a file, or a rasp, to forcibly separate cells. In some instances the loosening device also acts as the scraping device, in other instances the loosening device is used in conjunction with a scraping device.

The term “scrape” is defined as using mechanical means by which cells or groups of cells freed by loosening are collected in proximity to each other. Typical scraping devices include those having abrading (abrasive or cutting) surfaces, e.g., physical edges or, on a finer level, sandpaper or other such abrasive surfaces, which loosen and collect cells and cell groups. As mentioned above, some abrading surfaces can be used to both loosen and scrape cells.

The term “retain” means mechanical, chemical, or other means by which a collection of loosened cells or loosened groups of cells are held in proximity to one another such that actions including shaking, dropping, or moving of the sampling device displace a minimal number of the cells from the retention area. Examples of mechanical retention include using a spoon shaped surface that can be sealed with adhesive tape or a cover or using a tube or other vessel into which samples can be placed. Chemical means of retention include the use of adhesives. Typical adhesives include acrylates or conventional pressure sensitive adhesives. However, adhesives such as a surface of imitation gecko toe hairs that works using van der Waals forces as explained in http://www.nature.com/nsu/020826/020826-2.html are also suitable.

The device is useful for loosening, scraping, and retaining epidermal cell samples but not useful for loosening, scraping, and retaining body cavity tissue cell samples. In contrast to devices used for cell samples taken from a variety of body cavities, the present devices are designed to collect and store cells from the epidermis, a keratinized epithelial tissue. The body cavities typically contain softer tissue of non-keratinized epithelium. The “tougher” construction of the epidermis allows it to act as a protective barrier, and also makes it more difficult to obtain a cell or tissue sample. Thus, without the first step of aggressive action to loosen cells, it is unlikely that the existing devices would collect and securely retain enough cellular matter from the epidermis for subsequent nucleic acid, e.g., DNA, extraction and analysis.

### BRIEF DESCRIPTION OF THE DRAWINGS

FIG. 1 shows a side view of a first embodiment of a device of the present invention having a collection and scraping surface having one or more slanted knife edge projections.

FIG. 2 shows a front cross-sectional view along view II-II of FIG. 1.

FIG. 3 shows a top view of the skin cell scraping and collection device of FIG. 1.

FIG. 4 shows an exploded top view of a second embodiment of the present invention having a collection and scraping surface having one or more slanted knife edge projections and a sliding cover.

FIG. 5 shows a partial cross-sectional side view along view V-V along a longitudinal axis “L” of the embodiment of FIG. 4 in a closed position.

FIG. 6 shows a partial cross-sectional side view of the embodiment of FIG. 4 in an open position.

FIG. 7 shows a top view of a third embodiment of the present invention having a collection and scraping surface having one or more slanted knife edge projections and a hinged cover in an open position.

FIG. 8 shows a partial cross-sectional side view of the embodiment of FIG. 7 along view VIII-VIII with the hinged cover in the open position.

FIG. 9 shows a partial cross-sectional side view of the third embodiment of FIG. 7 with the hinged cover in a closed position.

FIG. 10 shows a front view of the cover of the embodiment of FIG. 7.

FIG. 11 shows a side view of a fourth embodiment of a device of the present invention having a collection and scraping surface having one row, or a series of rows, having slanted leading knife edge projections extending from the surface.

FIG. 12 shows a top view of the embodiment of FIG. 11.

FIG. 13 shows a partial cross-sectional side view of the embodiment of FIG. 11 modified to have a slidable cover.

FIG. 14 shows a partial cross-sectional side view of the embodiment of FIG. 11 modified to have a hinged cover.

FIG. 15 shows a side view of a fifth embodiment of a device of the present invention having a collection and scraping surface having a single blade extending from an angle from a plane to scrape off (loosen) and collect a surface skin cells sample.

FIG. 16 shows a partial cross-sectional side view of the embodiment of FIG. 15 modified to have a slidable cover.

FIG. 17 shows a partial cross-sectional side view of the embodiment of FIG. 15 modified to have a hinged cover

FIG. 18 shows a top view of a sixth embodiment of a device of the present invention having a stick made of a suitable material such as polystyrene or polycarbonate and a spoon-like hollow lined with abrasive.

FIG. 19 shows a side view the sixth embodiment of a device of the present invention having a stick made of a suitable material such as polystyrene or polycarbonate and a spoon-like hollow lined with abrasive.

FIG. 20 shows a top view of a modified version of the embodiment of FIG. 18 which includes a cover made of a pliable plastic that can be sealed with adhesive strips on its edges.

FIG. 21 shows a top view of the seventh embodiment of the present invention which is the embodiment of FIG. 18 modified to include a cap and a rim.

FIG. 22 shows a side view of the seventh embodiment of FIG. 21.

FIG. 23 shows a perspective view of an eighth embodiment of a device of the present invention in which the blade-like device is placed inside a removable cap.

FIG. 24 shows a bottom view of the embodiment of FIG. 23.

FIG. 25 shows an embodiment of a kit of the present invention.

### DETAILED DESCRIPTION OF THE PREFERRED EMBODIMENTS

The present invention provides methods, devices and kits capable of the three steps of loosening, scraping, and retaining the sample of epidermal (surface skin) cells in some form or fashion. The three steps (loosening, scraping, and retaining) can be applied simultaneously or they can be applied sequentially.

Loosening a surface skin cell sample is performed by rubbing a device having an abrasive or cutting surface, for example sandpaper, to abrade the surface of the skin or a knife-like or blade-like or rasp-like instrument to forcibly loosen cells, applying enzymes such as, but not restricted to, papain to break bonds between cells, or treating the epidermis with sonic energy to break cell bonds, or a combination of these techniques. The rubbing may be in a single direction or by a back and forth motion. The abrasive or cutting device would probably be a single use item. Thus, before use it would be in a sealed container (for example, the test tube) or individual foil or plastic sealed package (like an individual WET ONES moist towelette package). Examples of the abrasive or cutting device include sandpaper, a cheese grater-like surface having hollow open faced projections, or a surface having one or more rows having slanted leading projections with sharp edges extending from the surface.

The abrasive or cutting device can simply have an abrasive or cutting surface or it may have an abrasive or cutting surface with adhesive material on the surface to further hold the scraped off sample. For example, the present invention encompasses use of sandpaper or other substrate, provided with a layer of uniform abrasive, e.g., sand or diamond particles wherein the grit has a particle size in the range of 50 to 500 mesh, optionally further provided with adhesive to retain the cell sample. The abrasive or cutting surface can be made by any conventional means. If desired, the abrasive or cutting surface can be made by nanotechnology/micromachining. In some instances, the abrasive or cutting projections comprise microtines or microblades that extend 10 to 100 μm from a surface of the abrasive or cutting device.

Scraping and collecting the loosened sample is performed by rubbing the device having the abrasive or cutting surface on skin. Thus, in some instances the apparatus having the abrasive or cutting surface performs the steps of loosening, scraping and collecting the surface skin cell sample. Moreover, enzymes or sonic energy can be applied to the external skin surface prior to using the apparatus having the abrasive or cutting surface to enhance the loosening performed by the apparatus having the abrasive or cutting surface.

Retaining the sample is performed by sealing the device, that now includes the scraped off skin cell sample, with a cover. This cover may contain a desiccant. The device could be placed into a container for safekeeping. A typical container is a vapor barrier pouch with desiccant to store the cell sample for a long period of time. This overcomes drawbacks of refrigerated storage. Typical freezer temperatures, e.g. minus 5 degrees F., are insufficient to protect cells from fungus and other micro-organisms. Suitable vapor barrier pouches with desiccant are available from the manufacturers listed in TABLE 1.

TABLE 1

| Manufacturer of | | |
|---|---|---|
| Component | Component | Web Site |
| LPS Industries | Vapor barrier bag | www.lpsind.com |
| | and desiccant | |
| Sorbent Systems | Vapor barrier bag | www.sorbentsystems.com |
| | and desiccant | |
| PolyLam | Vapor barrier and | www.polylam.com |
| | desiccant | |

Typically, the present invention advantageously collects and stores the sample dry. In contrast, Whatman and Schleicher & Schuell have both been granted patents (described above) for long term storage of DNA bearing material but require that the sample be applied to the paper in a liquid form. However, if desired, the epidermal cells may be collected, suspended in liquid, and then transferred to a sheet of Whatman, Schleicher & Schuell or other filter paper.

The scraped off skin cells sample would then be processed for conventional DNA analysis and/or for biological analyses other than DNA analysis, for example, cytological, diagnostic or other scientific biochemical analyses.

For example, the DNA from the scraped off skin cells sample would be removed and analyzed by any conventional method. Removing could be done by, e.g., washing, the cells from the abrasive or cutting surface followed by collecting the DNA from these removed cells for analysis. In the alternative, the DNA could be removed from the cells while they still adhere to the abrasive or cutting surface or while they remain in the collection well. For example, the cells may be ruptured to remove their DNA followed by a typical DNA analysis. Typical DNA analyses include, but are not limited to, hybridization behavior, electrophoretic mobility, and nucleotide sequence determination, amplification of one or more DNA sequences by polymerase chain reaction (PCR), determination of the length and/or DNA sequence of the PCR amplified DNA. In one example, the DNA sequences amplified by PCR comprise one or more short tandem repeat (STR) loci. Examples of other scientific analyses include characterization of RNA, protein, lipids and other components of the epidermis.

The present invention also relates to a kit comprising the device for loosening, scraping and retaining surface skin cells as well as the vapor barrier pouch with desiccant, where the sample can be maintained without contamination.

A number of embodiments of devices suitable for being employed in the method of the present invention and/or being included in the kit of the present invention, are presented below.

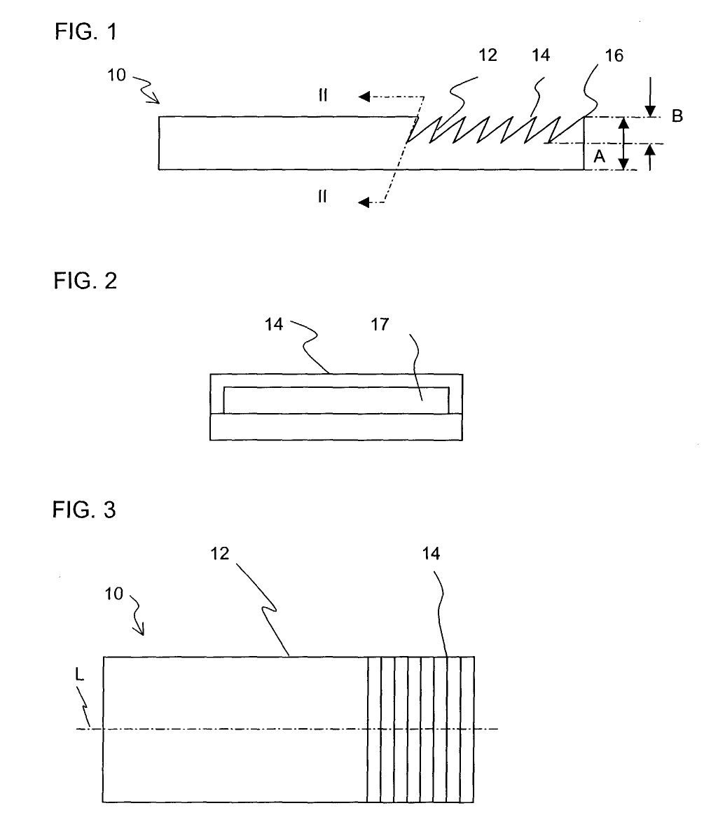

FIG. 1 shows a side view of a skin cell scraping and collection device 10 made of a suitable material such as polymer (e.g., polystyrene), metal, glass or ceramics. The device 10 has a collection and scraping surface 12 made of a polymer and having one or more slanted knife edge projections 14 having a height “B” and an end wall 16 having a height “A” and an appropriate degree of sharpness. The dimensions are selected such that they work to abrade and/or scrape off cells but not draw blood. For example, if the knife-edge projections are sufficiently sharp to cut through (slice) skin, then the height “B” is selected such that the device cannot penetrate the epidermis so far that it draws blood, e.g. about 10 to 100 μm. For example, the height “B” could be sufficient such that the blade could penetrate the stratum corneum without substantially damaging underlying layers. If the edge sharpness is sufficient to abrade the epidermis but not cut through the epidermis then the height “B” can be in the range from about 10 μm to about 1 centimeter, or typically about 100 μm to about 5 millimeters. End wall height “A” would be any practical dimension. Typical heights “B” range from about 10 μm to 5 mm.

If desired, the knife-edge projections 14 can be hollow and have an open portion 17 in the slanted wall under the edge 16 like a cheese grater as shown in FIG. 2. FIG. 2 is an end on view when viewing from near the end wall 16. If desired, the portions of the surface 12 in the valleys between the knife edges projections 14 may be coated with adhesive to assist in retaining the skin cell sample.

FIG. 3 shows a top view of the skin cell scraping and collection device 10. The knife-edge projections are arranged transverse to a longitudinal axis “L” of the device 10.

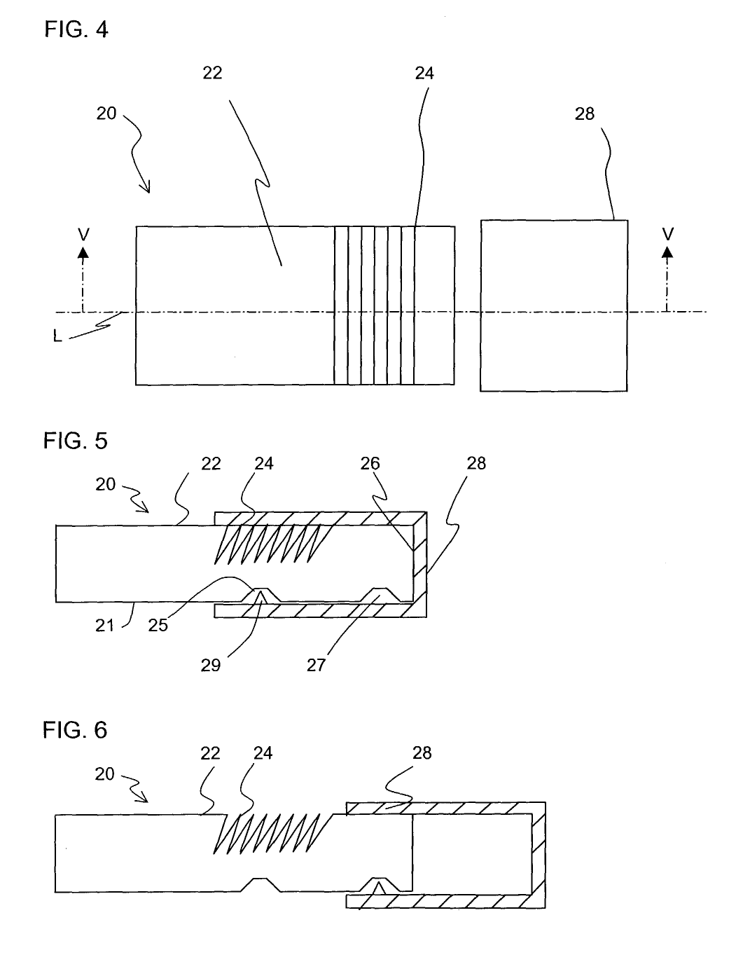

FIG. 4 shows an exploded top view a second embodiment of a device 20 of the present invention having a collection and scraping surface 22 having one or more slanted knife edge projections 24, an end wall 26 and a sliding cover 28.

FIG. 5 shows a partial cross-sectional side view of the embodiment of FIG. 4 with the sliding cover 28 in a closed position. FIG. 6 shows the embodiment of FIG. 4 with the sliding cover 28 in an open position.

The cover 28 slides longitudinally between the closed position (FIG. 5) and the open position (FIG. 6). The device 20 may be kept in the closed position before use to prevent contamination of the collected sample. However, the device 20 may be kept in the open position before use if it is stored in a sealed package before use to prevent contamination. The device 20 is kept in the closed position after use to prevent contamination of the collected sample. The side 21 of the device 20 opposed to the collection and scraping surface 22 has a first transverse groove 25 relatively distal to the end wall 26 and a second transverse groove 27 relatively proximal to the end wall 26. A projection, namely a transverse ridge 29, extends from an inside wall of the cover 28 into the cover 28 to engage the first transverse groove 25 to assist in maintaining the cover 28 in the closed position as shown in FIG. 5. The ridge 29 engages the second transverse groove 27 to assist in maintaining the cover 28 in the open position as shown in FIG. 6. Although the grooves 25, 27 are shown in FIGS. 5 and 6 to transverse the entire surface 21 of the device 20, grooves that do not transverse the entire surface 21 would also suffice. Likewise the ridge 29 may transverse across the entire side of the inner surface of the cover 28 from which the ridge 29 extends, or transverse across a portion of this side.

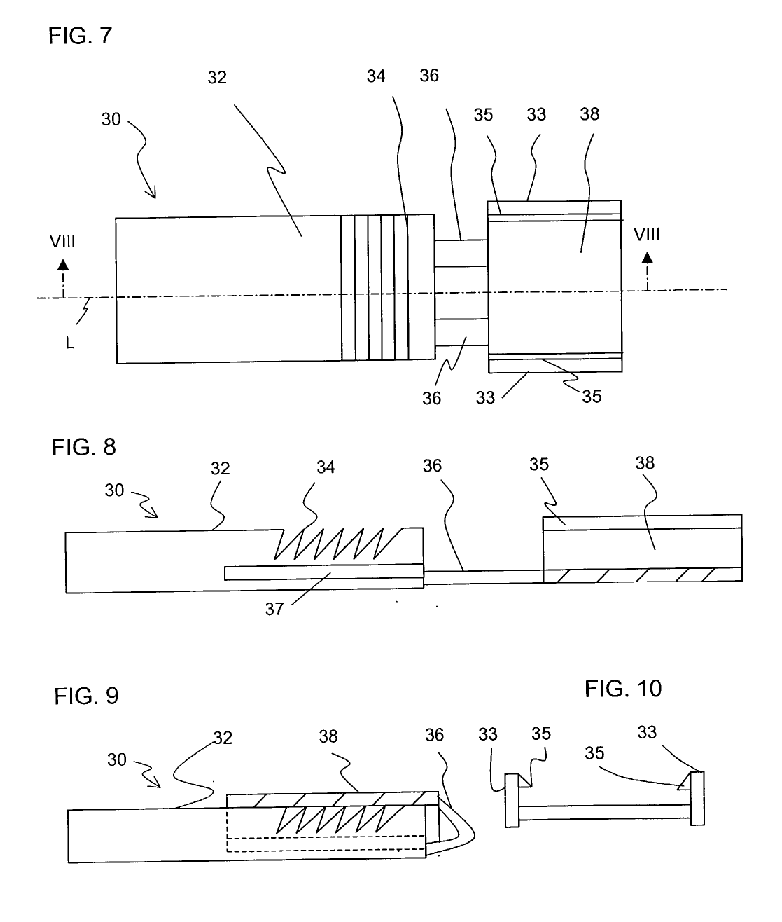

FIG. 7 shows a top view of a third embodiment of the device 30 of the present invention having a collection and scraping surface 32 having one or more slanted knife edge projections 34 and cover 38 held to the device 30 by a pair of flexible hinges 36.

FIG. 8 shows a partial cross-sectional side view of the third embodiment of FIG. 7 along view VIII-VIII wherein the hinged cover 38 is in an open position.

FIG. 9 shows a partial cross-sectional side view of the third embodiment of FIG. 7 wherein the hinged cover 38 is in a closed position.

The cover 38 may be held in the closed position by a friction fit. However, other means for holding the cover 38 in the closed position are also possible. For example, as shown in FIG. 10, each sidewall 33 of the cover 38 may have a respective ridge 35 to fit into a respective groove 37 on opposed sides of the device 30 (FIG. 8) to assist in holding the cover 38 in the closed position.

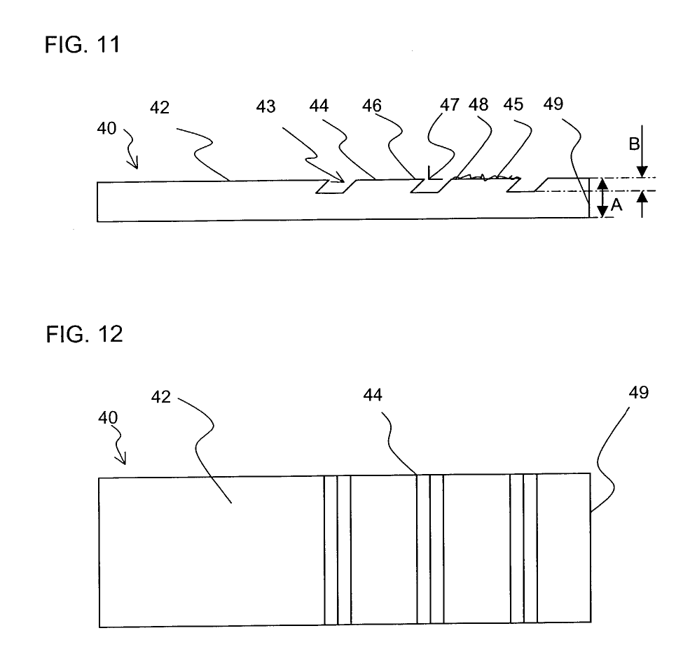

FIG. 11 shows a side view of a fourth embodiment of a skin cell scraping and collection device 40 made of a suitable material as described above for the first embodiment. The device 40 has a collection and scraping surface 42 comprising a plurality of rows 44 and an end wall 49 having a height “A”. Each row 44 has a slanted leading knife-edge projection 46 (extending a height “B”), a leading wall 47, a top wall 48 and a trailing wall 43. Typical dimensions for “A” and “B” of the second embodiment are as for the above described first embodiment. The top walls 48 of each knife-edge projection 46 may have a smooth surface or provide additional abrasive surfaces (e.g., optional abrasive surface 45). The additional abrasive surfaces are achieved by molding/machining the surface of the top walls 48 to be rough or by fixing a layer of abrasive onto the top walls 48.

A suitable abrasive surface is a single layer of uniform diamond or sand particles embedded on the surface of the top wall and having a grit ranging from 50 to 500 mesh fine grit crushed diamond, or a metal file surface or a rasp surface. If desired the abrasive surface may be a plurality of microprotrusions disclosed in U.S. Pat. No. 5,879,326 to Godshall et al. having a height sufficient to disrupt a layer of epidermis without cutting beyond a predetermined distance. If desired, the knife-edge projections 46 can be hollow and open in the slanted leading wall 47 like a cheese grater as explained above for the embodiment of FIG. 2. If desired, the walls 43 and 47 as well as the surface connecting them may be coated with adhesive to assist in retaining the retained skin cell sample. Optionally adhesive may be located in the valleys between the knife-edge projections 46. Moreover, if desired the embodiment of FIG. 11 may have a cover, for example like that of FIG. 5 or FIG. 10.

FIG. 12 shows a top view of the skin cell scraping and collection device 40 shown in FIG. 11.

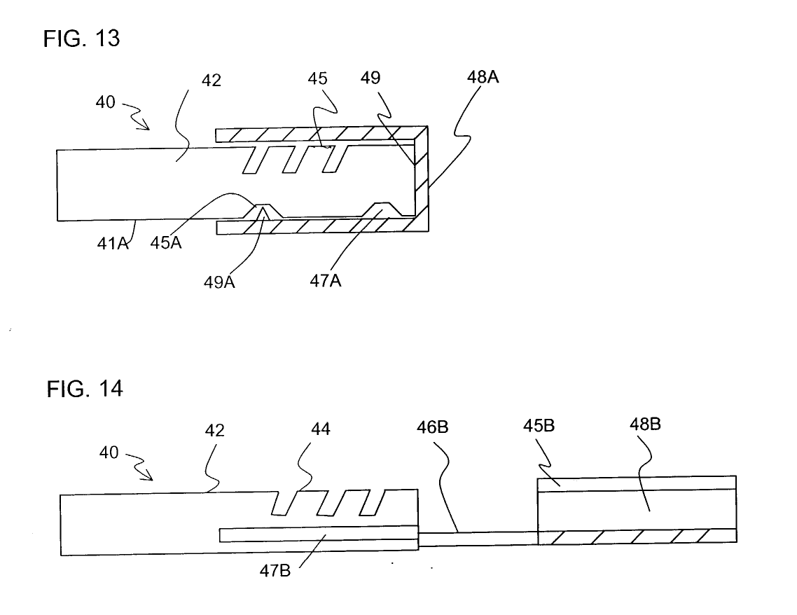

FIG. 13 shows a device 40 of FIG. 11 modified (as in the embodiment of FIG. 5) to have a slidable cover 48A. The cover 48A slides longitudinally between a closed position (FIG. 13) and an open position (not shown but easily to envision from FIG. 6). The device 40 may be kept in the closed position before use to prevent contamination of the collected sample. However, the device 40 may be kept in the open position before use if it is stored in a sealed package before use to prevent contamination. The device 40 of FIG. 13 is kept in the closed position after use to prevent contamination of the collected sample. A lower side 41A of the device 40 of FIG. 13 opposed to the collection and scraping surface 42 has a first transverse groove 45A relatively distal to the end wall 49 and a second transverse groove 47A relatively proximal to the end wall 49. A projection, namely a transverse ridge 49A, extends from an inside wall of the cover 48A into the cover 48A to engage the first transverse groove 45A to assist in maintaining the cover 48A in the closed position as shown in FIG. 13. The ridge 49A engages the second transverse groove 47A to assist in maintaining the cover 48A in the open position (not shown but easy to envision from FIG. 6). In this embodiment the grooves 45A, 47A transverse the entire surface 41A of the device 40. However, grooves that do not transverse the entire surface 41A would also suffice. Likewise the ridge 49A may transverse across the entire side of the inner surface of the cover 48A from which the ridge 49A extends, or transverse across a portion of this side.

FIG. 14 shows the embodiment of the device 40 of FIG. 11 modified to have a hinged cover 48B (as does the embodiment of FIG. 8) held to the device 40 by a pair of flexible hinges 46B (one shown by this side view) in an open position. FIG. 14 is a partial cross-sectional view as is FIG. 8. The cover 48B may be held in the closed position (not shown but easy to envision from FIG. 9) by a friction fit. However, other means for holding the cover 48B in the closed position are also possible. For example, as shown in FIG. 14, opposed sidewalls of the cover 48B may have a respective ridge 45B to fit into a respective groove 47B on opposed sides of the device 40 to assist in holding the cover 48 in the closed position.

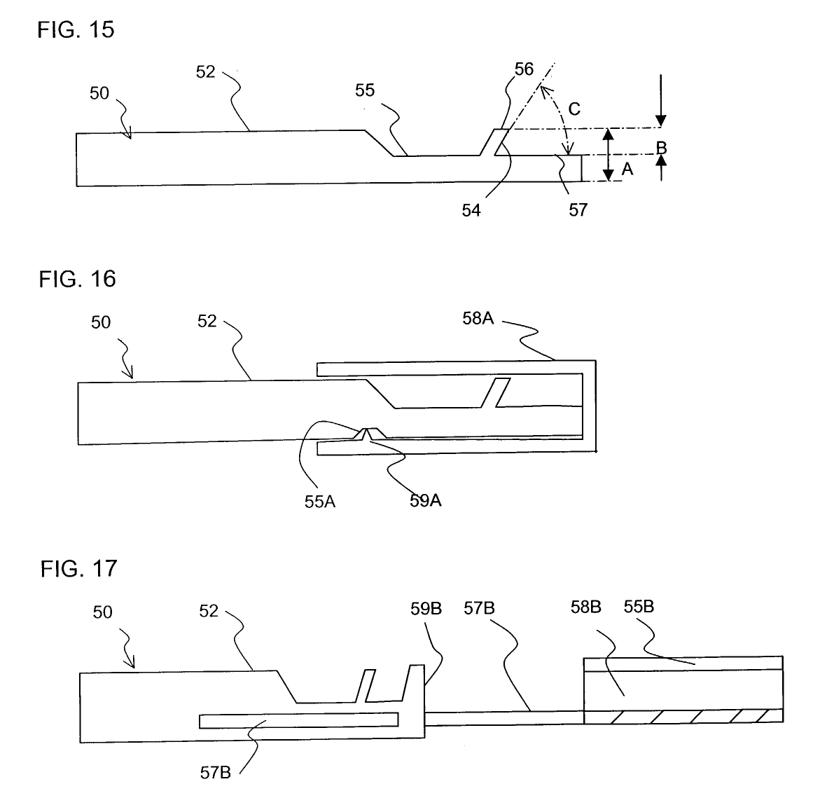

FIG. 15 shows a side view of a fifth embodiment of a skin cell scraping and collection device 50 made of a suitable material as described above for the first embodiment. The device 50 is much the same as the device 40 of FIG. 11. The device 50 has a thickness “A”, and a top wall surface 52 having a single blade 54 extending a height “B” from a plane having back portion 55 and front portion 57 to loosen, scrape off, and collect a surface (epidermal) skin cells sample. Relative to the front portion 57, the single blade 54 extends from the front portion 57 at an angle “C” which is typically in the range from about 30 to about 90 degrees, more typically about 45 to about 80 degrees. Optionally, a top wall 56 of the single blade 54 may have an abrasive surface and the front portion 57 may have an adhesive surface similar to the abrasive and adhesive surfaces mentioned above for device 40. The abrasive surface may be provided by molding/machining the surface to have a rough coat or by applying an abrasive coating to the top wall 56.

Moreover, FIG. 16 shows a modified version of the device 50 of FIG. 15 provided with a slidably removable cap 58A in a closed position. The cap 58A comprises a sleeve having opposed open and closed ends (much like the cap of FIG. 5) and may be held onto the device 50 in the closed position by a friction fit, and/or a transverse ridge 59A engaging with a transverse groove 55A. In this embodiment, the cap 58A comes completely off in the open position (not shown, but easy to envision from the exploded view of FIG. 4).

FIG. 17 shows the embodiment of the device 50 of FIG. 15 modified to have a hinged cover 58B (as does the embodiment of FIG. 8) held to the device 50 by a pair of flexible hinges 56B (one shown by this side view) in an open position. FIG. 17 is a partial cross-sectional view as is FIG. 8. The cover 58B may be held in the closed position by a friction fit. However, other means for holding the cover 48B in the closed position are also possible. For example, in a similar manner as described above for the embodiment of FIG. 8, opposed sidewalls of the cover 58B may have a respective ridge 55B to fit into a respective groove 57B on opposed sides of the device 40 to assist in holding the cover 58B in the closed position. To assist in forming a seal the device 50 of FIG. 17 can be modified to have an extended end wall 59B having the same height as height “A” shown in FIG. 15.

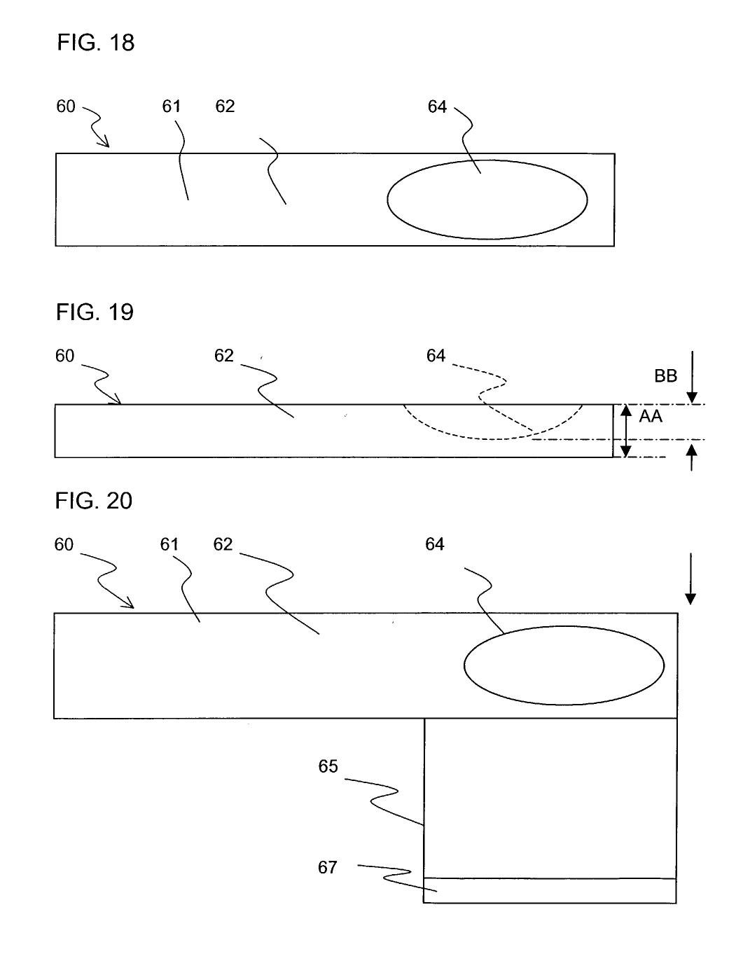

FIGS. 18 and 19 show a sixth embodiment of a device 60 of the present invention. Device 60 includes a stick 61 having a thickness “AA” made of a suitable material as described above for the first embodiment and having a top surface 62. A portion of the top surface 62 has an area 64 that has been hollowed out much as a spoon to a depth “BB”. Inside this hollowed out area 64 is an abrasive surface, typically made of the same surfaces and materials as described above for the top wall 48, 56. The user holds this stick 61 in his hand and rubs his thumb, or other body part, with a back and forth motion in the hollowed out portion 64 for a sufficient time to remove cells so that a sample is collected. The hollowed out portion 64 may contain adhesive to assist in retaining the sample. For example, if the abrasive surface is a file surface, adhesive may be provided in valleys between ridges of the file.

If desired to further secure the collected sample in the hollowed out area 64, a cap or tape or film is placed over the spoon area as further explained below.

FIG. 20 shows a top view of the device 60 of the sixth embodiment of FIG. 18 modified to include a tape or film 65 having an adhesive strip 67. After the cell sample has been deposited in the hollowed out depression 64, the tape or film 65 is wrapped over the hollowed out depression 64 to seal the sample. FIG. 20 shows the adhesive strip 67 as a single strip along one edge of the film 65. If desired, in an embodiment (not shown), the adhesive strip may extend about the entire perimeter of the tape or film 65. In FIG. 20 the tape is attached to the stick 61 before use and folded over the hollowed out depression 64 after the sample is in the depression 64. However, in alternate embodiments (not shown) the tape or film can be provided to have the adhesive strip, two parallel adhesive strips, or the adhesive perimeter and be initally not attached to the stick 61 and placed over the depression 64 after the sample is in the depression 64.

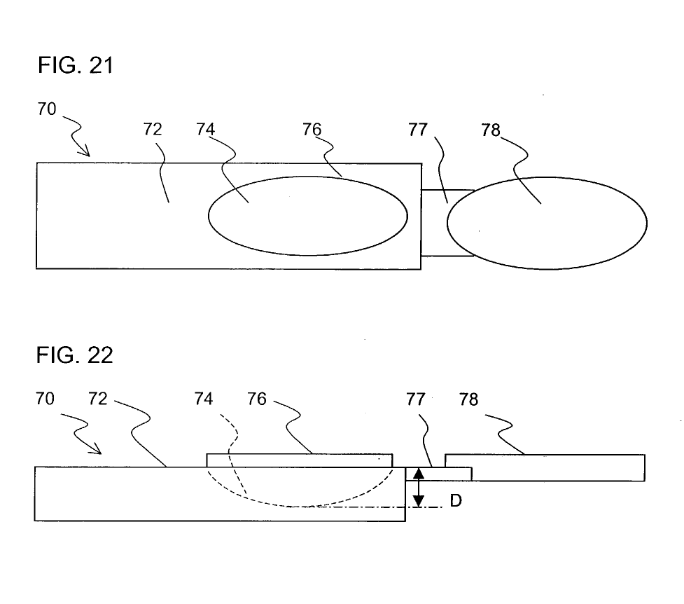

FIGS. 21 and 22 show top and side views respectively of a seventh embodiment of a device 70 of the present invention. Device 70 is similar to device 60 of FIG. 18 but is modified to include a cap 78 attached to the device 70 by a flexible neck (hinge) 77. This device 70 also has a hollowed out depression 74 having a depth D and a rim 76 protruding from a surface 72 of the device 70. After the cell sample has been deposited in the hollowed out depression 74, the cap 78 is snapped to the rim 76 of the hollowed out depression 74 to seal the sample.

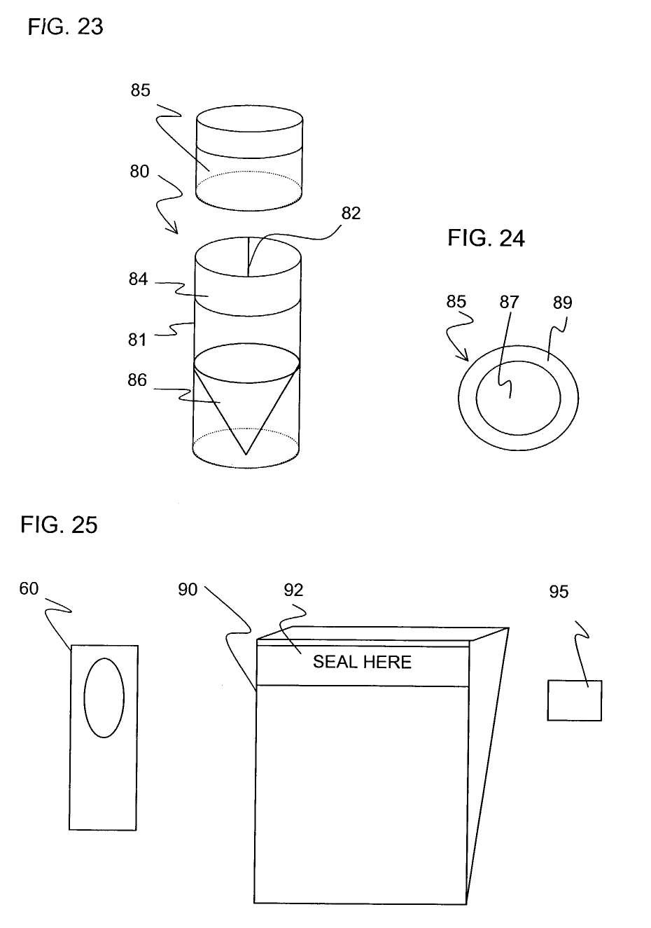

FIG. 23 shows a perspective view of an eighth embodiment of a device 80 of the present invention in which a metal blade-like cutting edge 82 is placed inside a removable cap 84. This cap 84 sits securely at the top of a cylinder 81 (or pen-like apparatus). Just below this blade-containing cap 84 is a collection well 86.

In particular, the metal blade-like cutting edge 82 sits in the removable cap 84 in the top of the cylinder 81. This removable cap 84 sits just above the collection well 86. Once the sample has been taken, a second cap, namely a screw cap 85, fits securely over the first cap 84 to minimize contamination and moisture transfer. Thus, a seal between the screw cap 85 and cylinder 81 is formed to minimize introduction of moisture into the sample. In FIG. 23 the cooperating threads of an upper portion of cylinder 81 and the screw cap 85 are not shown for clarity.

The second (threaded screw) cap 85, possibly contains desiccant. FIG. 24 shows a bottom view of the interior of the screw cap 85 wherein a desiccant 87 is held against the inside roof of the screw cap 85 by a silicone “O” ring 89. A typical material of construction for the cylinder 81 is 1.5 ml polypropylene tube having a threaded end to engage the threading of the screw cap 85. Typically, such a threaded tube is 0.5 to 2 cm, e.g. 1 cm, in diameter; and about 2 to 6 cm, e.g. 4 cm. tall (including the removable cap 84). The screw cap 85 is typically about 0.25 to 1.5 cm, e.g. 0.5 cm, tall. Other dimensions may also be suitable.

After a sample has been taken, the sample can be secured in a sterile bag or other container to be transported to the destination. If long-term storage will be required, bags constructed with a vapor barrier material such as a metallized film should be used. When an appropriately sized desiccant is placed in such a bag with the sample, and the bag is completely sealed by heat or another mechanism, the longevity of the sample will be substantially increased due to the reduction of water vapor in the bag. By controlling the amount of water within the sample storage environment, the microorganisms whose activity relies upon water is minimized. Thus, the effective lifetime of the sample is increased.

FIG. 25 shows an embodiment of a kit of the present invention. The kit includes a sampling device, e.g., device 60, a vapor barrier bag 90 and a desiccant packet 95. The sizing of the desiccant packet 95 depends upon the length of time the user wishes to store the sample in a controlled environment, the size and quality of the vapor barrier bag 90 as well as the type of the contents of the bag 90. For instance, paper would require more desiccant than plastic since paper attracts and retains water more easily. The sampling device 60 and the desiccant bag 95 are inserted into the open end 92 of the vapor barrier bag 90 and the bag 90 is then sealed. Typically the open end 92 of the vapor barrier bag 90 is sealed by heat sealing or other suitable means, e.g., chemical or adhesives.

An alternative storage embodiment (not shown) collects cells using a collecting device of the present invention, then suspends the collected cells in fluid and then places the collected suspended cells onto the Whatman or Schleicher & Schuell papers.

Processing the Sample

The sample may be used in any of a variety of biological tests for target components. Examples of such tests include, but are not limited to DNA testing, cytological tests and/or diagnostic testing. Of course a given test may simultaneously fill more than one of these categories.

The epidermal sample can be processed to extract and analyze various components of the epidermis. For example, the DNA in the sample can be extracted and analyzed. For example, the cells can be resuspended in a buffer and the DNA extracted using: commercially available kits such as, but not limited to, DNA IQ and QuickAmp Extraction; proteases such as Proteinase K, detergents such as sodium dodecyl sulfate (SDS) and chaotropic agents such as urea or guanidine and diatomceous earth or silica particles; organic solvent extraction such as phenol/chloroform and DNA precipitation.

Some examples of subsequent DNA analyses include, but are not limited to, Polymerase Chain Reaction followed by gel electrophoresis; PCR followed by STR fragment analysis; PCR followed by DNA sequencing; Southern Blot analysis; whole genome amplification or replication followed by DNA sequencing or Southern Blot analysis.

Typical diagnostic tests include assaying steps such as immunofluorescent staining and/or staining of the sample with any of a plurality of different conjugated antibodies or anti-sera.

It should be apparent that embodiments other than those described above come within the spirit and scope of the present invention. Thus, the present invention is not limited by the above-provided description but rather is defined by the following claims.

We claim:

1. A method for obtaining epidermal skin cells from a test subject comprising the steps of:
loosening the epidermal skin cells and/or other nucleic acid bearing material taken from the surface of the epidermis to form loosened epidermal skin cells and/or other DNA bearing material taken from the surface of the epidermis; and
scraping off and collecting a sample of the loosened epidermal skin cells and/or other DNA bearing material taken from the surface of the epidermis by rubbing a scraping and collecting apparatus having an abrasive or cutting surface on epidermal skin; and
sealing the collected epidermal skin cell sample.

2. The method of claim 1, wherein the sample is sufficient for genetic analysis of nucleic acid.

3. The method of claim 1, wherein the abrasive or cutting surface, that now includes the scraped off sample, is placed into a sealable container.

4. The method of claim 1, further comprising extracting nucleic acid from the sample on the abrasive or cutting surface.

5. The method of claim 1, further comprising separating the sample from the abrasive or cutting surface and then extracting nucleic acid from the separated sample.

6. The method of claim 1, further comprising subjecting the collected sample to cytological or other scientific analyses.

7. The method of claim 1, wherein the epidermal skin cells are loosened from the epidermis of the hand, foot, arm, or footpad.

8. The method of claim 1, wherein loosening comprises applying enzymes to break bonds between epidermal skin cells.

9. The method of claim 1, wherein loosening comprises applying sonic energy to break bonds between epidermal skin cells.

10. The method of claim 1, wherein the loosening comprises rubbing an abrasive surface on the epidermal skin to abrade the surface of the outer skin.

11. The method of claim 1, wherein the loosening comprises rubbing an abrasive surface comprising adhesive material on the abrasive surface to further hold the scraped off sample.

12. The method of claim 1, wherein loosening comprises rubbing sandpaper on the epidermal skin to abrade the surface of the outer skin.

13. The method of claim 1, wherein loosening comprises rubbing a device comprising a sharp edge on the epidermal skin to forcibly separate epidermal skin cells, wherein the device comprising a sharp edge is also the scraping and collecting apparatus.

14. The method of claim 1, wherein loosening comprises rubbing a device comprising an abrasive surface on the surface skin to forcibly separate surface skin cells, wherein the device comprising the abrasive surface is also the scraping and collecting apparatus.

15. The method of claim 1, wherein the abrasive or cutting surface comprises an adhesive coating.

16. The method of claim 1, wherein the scraping and collecting apparatus comprises a stick having a surface having longitudinally opposed ends, wherein the surface defines a hollowed out depression having an abrasive surface;
wherein the test subject holds this stick in his or her hand and rubs his or her thumb, or other body part, on the abrasive surface of the hollowed out depression for a time sufficient to loosen and scrape off surface skin cells.

17. The method of claim 16, further comprising placing a cap or tape or film over the hollowed out depression after the loosening and scraping off of the surface skin cell sample to secure the sample in the hollowed out depression.

18. The method of claim 1, further comprising placing the sampling device inside a vapor barrier bag, with a desiccant, and then sealing the bag.

19. The method of claim 1, wherein the sample is suspended in a liquid, and then the suspended sample is applied to a paper, prior to being sealed.

20. A method for biological analysis, comprising the steps of:
taking a sample of epidermal skin cells of a test subject by use of the method of claim 1;
extracting nucleic acid and/or other target components from the collected sample; and
measuring physical or chemical properties of the nucleic acid and/or other target component.

21. The method of claim 20, wherein the biological analysis is genetic analysis.

22. An apparatus for obtaining epidermal skin cells and/or other nucleic acid bearing material from a test subject comprising:
means for loosening the epidermal skin cells to form loosened epidermal skin cells selected from the group consisting of enzymes that break bonds between epidermal skin cells and means for generating sonic energy to break epidermal skin cell bonds; and
an abrading surface for scraping off and collecting a sample of the loosened epidermal skin cells by rubbing.

23. The apparatus of claim 21, wherein the sample is sufficient for biological analysis.

24. The apparatus of claim 21, wherein loosening means comprises at least one enzyme.

25. The apparatus of claim 21, wherein loosening comprises the means for generating sonic energy to break surface skin cell bonds.

26. An apparatus for obtaining epidermal skin cells from a test subject comprising:
an abrading surface comprising an abrasive surface and/or a cutting edge for loosening the epidermal skin cells to form loosened surface skin cells and scraping off a sample of the loosened surface skin cells by rubbing a portion of epidermis on the abrasive surface or cutting edge; and
adhesive on and/or adjacent the abrading surface for collecting the sample of the loosened surface skin cells.

27. The apparatus of claim 26, comprising a top surface, wherein the abrading surface comprises the cutting edge extending from the top surface, wherein the cutting edge has a longitudinal axis transverse to the longitudinal axis of the apparatus.

28. The apparatus of claim 27, comprising the abrasive surface.

29. An apparatus for obtaining epidermal skin cells from a test subject comprising:
an abrading surface comprising an abrasive surface and/or a cutting edge for loosening the epidermal skin cells to form loosened surface skin cells and scraping off a sample of the loosened surface skin cells by rubbing a portion of epidermis on the abrading surface; and a cover for covering the abrading surface in a closed position.

30. The apparatus of claim 29, wherein the cover comprises a slidable cover for covering the abrading surface in a closed position.

31. The apparatus of claim 29, wherein the cover is attached to the apparatus by a flexible hinge.

32. The apparatus of claim 29, comprising the cutting edge, wherein the cutting edge comprises a sharp metal blade-like cutting edge placed inside a first removable cap, the first removable cap sitting securely at the top of a cylinder, just below the blade-containing first removable cap is a collection well, and the cover comprises a second cap for fitting securely over the first removable cap to minimize contamination and moisture transfer.

33. The apparatus of claim 32, wherein the second removable cap contains desiccant.

34. An apparatus for obtaining epidermal skin cells from a test subject comprising:
a collection and scraping surface comprising a cutting edge for loosening the epidermal skin cells to form loosened epidermal skin cells and scraping off a sample of the loosened epidermal skin cells by rubbing a portion of epidermis on the cutting edge in a direction transverse to a longitudinal axis of the cutting edge; and
wherein the cutting edge is slanted relative to a longitudinal axis of the collection and scraping surface, wherein the longitudinal axis of the cutting edge is transverse relative to the longitudinal axis of the collection and scraping surface, and the cutting edge has a leading wall, a top wall and a trailing wall, wherein the top wall has an abrasive surface.

35. The apparatus of claim 34, wherein the scraping and collecting apparatus comprises a stick having a surface having longitudinally opposed ends, wherein the surface defines a hollowed out depression having an abrasive surface;
wherein the test subject holds the stick in his or her hand and rubs his or her thumb, or other body part, in the hollowed out depression for a time sufficient to loosen and scrape off epidermal skin cells.

36. The apparatus of claim 34, further comprising a cover for covering over the hollowed out depression after the loosening and scraping off of the epidermal skin cell sample to secure the sample in the hollowed out depression.

37. An apparatus for obtaining epidermal skin cells from a test subject comprising:
a base having a surface having longitudinally opposed ends, wherein the surface defines a hollowed out depression having an abrasive surface;
wherein the hollowed out depression is sized for a test subject to rub his or her thumb, or other body part, in the hollowed out depression for a time sufficient to loosen and scrape off surface skin cells.

38. The apparatus of claim 37, further comprising a cover for covering over the hollowed out depression after the loosening and scraping off of the epidermal skin cell sample to secure the sample in the hollowed out depression.

39. The apparatus of claim 38, wherein the cover is selected from the group consisting of a cap attached to the base, an adhesive strip attached to the base, and an adhesive strip attachable to the base.

40. A kit for obtaining surface skin cells from a test subject comprising the apparatus of claim 22; and a sealable container.

41. A kit for obtaining surface skin cells from a test subject comprising the apparatus of claim 26; and a sealable container.

42. The kit of claim 41, wherein the sealable container comprises a vapor barrier bag, and the apparatus comprises a desiccant.

43. A kit for obtaining surface skin cells from a test subject comprising the apparatus of claim 29; and a sealable container.

44. The kit of claim 43, wherein the sealable container comprises a vapor barrier bag, and the apparatus comprises a desiccant.

45. A kit for obtaining surface skin cells from a test subject comprising the apparatus of claim 34; and a sealable container.

46. The kit of claim 45, wherein the sealable container comprises a vapor barrier bag, and the apparatus comprises a desiccant.

47. A kit for obtaining surface skin cells from a test subject comprising the apparatus of claim 37; and a sealable container.

48. The kit of claim 47, wherein the sealable container comprises a vapor barrier bag, and the apparatus comprises a desiccant.

49. An method for the long-term storage of skin cells for DNA sampling comprising:
storing a sample of epidermal cells of a test subject in a sealable container in the presence of a desiccant.

50. The method of claim 49, wherein the sealable container comprises a vapor barrier bag and the desiccant.
